(19) United States
(12) Patent Application Publication
(10) Pub. No.: US 2008/0139404 A1
(43) Pub. Date: Jun. 12, 2008

(54) Residual Enzyme Assays

(75) Inventors: Mads Eskelund Bjornvad, Frederiskberg (DK); Abel Gernot, Copenhagen (DK)

Correspondence Address: NOVOZYMES NORTH AMERICA, INC., 500 FIFTH AVENUE, SUITE 1600, NEW YORK, NY, 10110, US

(73) Assignee: NOVOZYMES A/S, Bagsvaerd (DK)

(21) Appl. No.: 11/628,146

(22) PCT Filed: Jun. 7, 2005
(86) PCT No.: PCT/DK05/00374

§ 371 Date: Nov. 29, 2006

(30) Foreign Application Priority Data
Jun. 7, 2004 (DK) PA 2004 00884

(51) Int. Cl. C40B 30/08; C12Q 1/00; C12Q 1/48; C12Q 1/34; C12Q 1/26; C40B 30/10

(52) U.S. Cl. 506/11; 435/4; 435/15; 435/18; 435/25; 506/12

(57) ABSTRACT

The present invention relates to a method for measuring the amount of residual enzyme on a textile comprising measuring the activity of the enzyme, wherein the textile has been contacted with the enzyme and subsequently rinsed prior to measuring the enzyme activity and to a method for screening a library of polypeptides for an enzyme of interest comprising testing the library in said method.

### FIELD OF THE INVENTION

The present invention relates to a method for measuring the amount of residual enzyme on a textile and a method for screening a library of polypeptides for an enzyme of interest comprising using this method.

### BACKGROUND OF THE INVENTION

Different enzymes, such as proteases, lipases and carbohydrases are often used within the detergent industry where they are typically contacted with the clothes during the process of washing as a component of the detergent and then subsequently removed when the clothes are rinsed. The interaction between the enzyme and the clothes and/or stains present at the clothes may depending on the particular enzyme be so strong that the enzyme is still present at the clothes after it has been rinsed.

Generally new and/or improved enzymes may be identified by screening libraries of polypeptides in an assay capable of testing the function of the enzyme under certain conditions. Often the ability to identify new and/or improved enzymes in such a library depends on the quality of the assay, e.g. the robustness of the assay and/or how well it mimics those conditions one wish the identified enzyme should be capable of functioning at.

The present invention provides methods for measuring the amount of residual enzyme on a textile.

### SUMMARY OF THE INVENTION

The invention provides a method for measuring the amount of residual enzyme on a textile comprising measuring the activity of the enzyme, wherein the textile has been contacted with the enzyme and subsequently rinsed prior to measuring the enzyme activity.

Furthermore, the present invention also provides a method for screening a library of polypeptides for an enzyme of interest comprising
a) measuring the amount of residual enzyme on a textile comprising measuring the activity of said enzyme, wherein the textile has been contacted with the library of polypeptides and subsequently rinsed prior to measuring said activity
b) selecting an enzyme of interest

### DETAILED DESCRIPTION OF THE INVENTION

### Enzyme/Enzyme of Interest

The enzyme/enzyme of interest may belong to a known class of enzymes, or it may be of an unknown enzyme class, e.g. an enzyme having a desired functional activity but not necessarily belonging to a known enzyme class. As used herein, the term “enzyme class” (E.C.) refers to the internationally recognized enzyme classification system, Recommendations (1992) of the Nomenclature Committee of the International Union of Biochemistry and Molecular Biology, Academic Press, Inc., 1992.

For example the enzyme/enzyme of interest may belong to one of the following classes: oxidoreductases (EC 1.-.-.-), transferases (EC 2.-.-.-), hydrolases (EC 3.-.-.-), lyases (EC 4.-.-.-), isomerases (EC 5.-.-.-) and ligases (EC 6.-.-.-).

### Oxidoreductases

Examples of oxidoreductases include peroxidases (EC 1.11.1), laccases (EC 1.10.3.2) and glucose oxidases (EC 1.1.3.4).

### Transferases

Examples of transferases may be transferases belonging to any of the following sub-classes:
a) Transferases transferring one-carbon groups (EC 2.1);
b) Transferases transferring aldehyde or ketone residues (EC 2.2); acyltransferases (EC 2.3);
c) Glycosyltransferases (EC 2.4);
d) Transferases transferring alkyl or aryl groups, other than methyl groups (EC 2.5); and
e) Transferases transferring nitrogeneous groups (EC 2.6).

In particular the transferase may be a transglutaminase (protein-glutamine gamma-glutamyltransferase; EC 2.3.2.13).

### Hydrolases

Examples of hydrolases include: Carboxylic ester hydrolases (EC 3.1.1.-). In particular it may be a lipolytic enzyme, i.e. an enzyme which can hydrolyze an ester bond. Such enzymes include, for example, lipases, such as triacyl-glycerol lipase (EC 3.1.1.3), lipoprotein lipase (EC 3.1.1.34), monoglyceride lipase (EC 3.1.1.23), lysophospholipase, ferulic acid esterase and esterase (EC 3.1.1.1, EC 3.1.1.2). The numbers in parentheses are the systematic numbers assigned by the Enzyme Commission of the International Union of Biochemistry in accordance with the type of the enzymatic reactivity of the enzyme.

The lipolytic enzyme may be prokaryotic, particularly a bacterial enzyme, e.g. from Pseudomonas. Examples are Pseudomonas lipases, e.g. from P. cepacia (U.S. Pat. No. 5,290,694, pdb file 1OIL), P. glumae (N Frenken et al. (1992), Appl. En-vir. Microbiol. 58 3787-3791, pdb files 1TAH and 1QGE), P. pseudoalcaligenes (EP 334 462) and Pseudomonas sp. strain SD 705 (FERM BP-4772) (WO 95/06720, EP 721 981, WO 96/27002, EP 812 910). The P. glumae lipase sequence is identical to the amino acid sequence of Chromobacterium viscosum (DE 3908131 A1). Other examples are bacterial cutinases, e.g. from Pseudomonas such as P. mendocina (U.S. Pat. No. 5,389,536) or P. putida (WO 88/09367).

Alternatively, the lipolytic enzyme may be eukaryotic, e.g. a fungal lipolytic enzyme such as lipolytic enzymes of the Humicola family and the Zygomycetes family and fungal cutinases.

Examples of fungal cutinases are the cutinases of Fusarium solani pisi (S. Longhi et al., Journal of Molecular Biology, 268 (4), 779-799 (1997)) and Humicola insolens (U.S. Pat. No. 5,827,719).

The Humicola family of lipolytic enzymes consists of the lipase from H. lanuginosa strain DSM 4109 and lipases having more than 50% homology with said lipase. The lipase from H. lanuginosa (synonym Thermomyces lanuginosus) is described in EP 258 068 and EP 305 216 and has the amino acid sequence shown in positions 1-269 of SEQ ID NO: 2 of U.S. Pat. No. 5,869,438.

The Humicola family also includes the following lipolytic enzymes: lipase from Penicillium camembertii (P25234), lipase/phospholipase from Fusarium oxysporum (EP 130064, WO 98/26057), lipase from F. heterosporum (R87979), lyso-phospholipase from Aspergillus foetidus (W33009), phospholipase A1 from A. oryzae (JP-A 10-155493), lipase from A. oryzae (D85895), lipase/ferulic acid esterase from A. niger (Y09330), lipase/ferulic acid esterase from A. tubingensis (Y09331), lipase from A. tubingensis (WO 98/45453), lysophospholipase from A. niger (WO 98/31790), lipase from F. solanii having an isoelectric point of 6.9 and an apparent molecular weight of 30 kDa (WO 96/18729).

The Zygomycetes family comprises lipases having at least 50% homology with the lipase of Rhizomucor miehei (P19515). This family also includes the lipases from Absidia reflexa, A. sporophora, A. corymbifera, A. blakesleeana, A. griseola (all described in WO 96/13578 and WO 97/27276) and Rhizopus oryzae (P21811). Numbers in parentheses indicate publication or accession to the EMBL, GenBank, GeneSeqp or Swiss-Prot databases.

Other relevant hydrolases include but are not limited to phytases (EC 3.1.3.-), e.g. 3-phytases (EC 3.1.3.8) and 6-phytases (EC 3.1.3.26); glycosidases (EC 3.2, which fall within a group denoted herein as “carbohydrases”), such as alpha-amylases (EC 3.2.1.1); peptidases (EC 3.4, also known as proteases); and other carbonyl hydrolases. Other hydrolases include xyloglucanase, arabinase, rhamno-galactoronase, pectinases, ligninases (for example polyphenol hydrolase).

Examples of relevant proteases (E.C. 3.4) include but are not limited to those of animal, vegetable or microbial origin or chemically modified or protein engineered mutants. The protease may be a serine protease or a metallo protease, particularly an alkaline microbial protease or a trypsin-like protease. Examples of alkaline proteases are subtilisins, especially those derived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g. of porcine or bovine origin) and the Fusarium protease described in WO 89/06270 and WO 94/25583.

Examples of commercially available protease enzymes include Alcalase™, Savinase™, Primase™, Duralase™, Esperase™, and Kannase™ (Novozymes A/S), Maxatase™, Maxacal™, Maxapem™, Properase™, Purafect™, Purafect OxP™, FN2™, and FN3™ (Genencor International Inc.).

In the present context, the term “carbohydrase” is used to denote not only enzymes capable of breaking down carbohydrate chains (e.g. starches) of especially five- and six-membered ring structures (i.e. glycosidases, EC 3.2), but also enzymes capable of isomerizing carbohydrates, e.g. six-membered ring structures such as D-glucose to five-membered ring structures such as D-fructose.

Carbohydrases of relevance include the following (EC numbers in parentheses): alpha-amylases (3.2.1.1), beta-amylases (3.2.1.2), glucan 1,4-alpha-glucosidases (3.2.1.3), cellulases (3.2.1.4), endo-1,3(4)-beta-glucanases (3.2.1.6), endo-1,4-beta-xylanases (3.2.1.8), dextranases (3.2.1.11), chitinases (3.2.1.14), polygalacturonases (3.2.1.15), lysozymes (3.2.1.17), beta-glucosidases (3.2.1.21), alpha-galactosidases (3.2.1.22), beta-galactosidases (3.2.1.23), mannanase (3.2.1.25), amylo-1,6-glucosidases (3.2.1.33), xylan 1,4-beta-xylosidases (3.2.1.37), glucan endo-1,3-beta-D-glucosidases (3.2.1.39), alpha-dextrin endo-1,6-alpha-glucosidases (3.2.1.41), sucrose alpha-glucosidases (3.2.1.48), glucan endo-1,3-alpha-glucosidases (3.2.1.59), glucan 1,4-beta-glucosidases (3.2.1.74), glucan endo-1,6-beta-glucosidases (3.2.1.75), endo-1,4-beta-mannanase (3.2.1.78), arabinan endo-1,5-alpha-L-arabinosidases (3.2.1.99), endo-1,6-beta-mannanase (3.2.1.101), lactases (3.2.1.108), chitosanases (3.2.1.132) and xylose isomerases (5.3.1.5).

However, enzymes not yet classified may also be relevant for the present invention. The enzyme/enzyme of interest may also be a variant of a known enzyme, wherein the term “variant” is to be understood as an enzyme which differs from another enzyme, typically a known enzyme generally called a parent enzyme, with regard to at least one amino acid position.

### Library of Polypeptides

The present invention also relates to a method for screening a library of polypeptides for an enzyme of interest. In the context of the present invention the term “library of polypeptides” is to be understood as a collection of at least two different polypeptides; i.e. at least two polypeptides which differ at one or more amino acid positions, e.g. the number of amino acids in the polypeptides may be different or the amino acid(s) at a particular position may be different.

Typically, the library of polypeptides may be prepared by introducing a library of nucleic acid sequences encoding the library of polypeptides into a host cell capable of expressing the polypeptides. Due to the genetic degeneracy the number of different nucleic acid sequences in said library may be higher than the number of different polypeptides.

In particular said library of nucleic acid sequences may encode variants of a parent enzyme; i.e. polypeptides which differ at, at least one amino acid position compared to a parent enzyme. Thus the screening method may be used to screen for variants of a parent enzyme. Such variants may be produced by e.g. random mutagenesis or site-directed mutagenesis of a parent enzyme or other methods known to a person skilled in the art. Thus in a particular embodiment the library of polypeptides may be a library of variants of parent enzyme. Examples of suitable parent enzymes include but are not limited to those mentioned above in the section of enzymes. In particular the parent enzyme may be a lipolytic enzyme e.g. a lipase from Humicola, e.g. H. lanuginosa, or Pseudomonas or Bacillus.

In another embodiment said library of nucleic acid sequences may encode polypeptides derived from one or a number of different organisms. Thus the method may be used to screen one or a number of different organisms for expression of a lipolytic enzyme activity.

In another embodiment the library of polypeptides may be prepared by synthesizing the polypeptides.

Methods for preparing a library of nucleic acid sequences, introducing it into a host cell, expressing the polypeptides encoded by said library of polypeptides in the host cells and methods for synthesizing polypeptides are well known to a person skilled in the art and may e.g. be found in “Molecular cloning: A laboratory manual”, Sambrook et al. (1989), Cold Spring Harbor lab., Cold Spring Harbor, N.Y.; Ausubel, F. M. et al. (eds.); “Current protocols in Molecular Biology”, John Wiley and Sons, (1995); Harwood, C. R., and Cutting, S. M. (eds.); “Molecular Biological Methods for Bacillus”, John Wiley and Sons, (1990); “DNA Cloning: A Practical Approach, Volumes I and II”, D. N. Glover ed. (1985); “Oligonucleotide Synthesis”, M. J. Gait ed. (1984); “Nucleic Acid Hybridization”, B. D. Hames & S. J. Higgins eds (1985); “Transcription And Translation”, B. D. Hames & S. J. Higgins, eds. (1984); “Animal Cell Culture”, R. I. Freshney, ed. (1986); “Immobilized Cells And Enzymes”, IRL Press, (1986); “A Practical Guide To Molecular Cloning”, B. Perbal, (1984).

### Textile

In the context of the present invention the term “textile” includes fabrics, garments, and yarns.

Fabric can be constructed from fibers by weaving, knitting or non-woven operations. Weaving and knitting require yarn as the input whereas the non-woven fabric is the result of random bonding of fibers (paper can be thought of as non-woven). In the present context, the term “fabric” is also intended to include fibers and other types of processed fabrics.

Woven fabric is constructed by weaving “filling” or weft yarns between wrap yarns stretched in the longitudinal direction on the loom. The wrap yarns must typically be sized before weaving in order to lubricate and protect them from abrasion at the high speed insertion of the filling yarns during weaving. The filling yarn can be woven through the warp yarns in a “over one—under the next” fashion (plain weave) or by “over one—under two” (twill) or any other myriad of permutations. Strength, texture and pattern are related not only to the type/quality of the yarn but also the type of weave. Generally, dresses, shirts, pants, sheetings, towels, draperies, etc. are produced from woven fabric.

Knitting is forming a fabric by joining together interlocking loops of yarn. As opposed to weaving which is constructed from two types of yarn and has many “ends”, knitted fabric is produced from a single continuous strand of yarn. As with weaving, there are many different ways to loop yarn together and the final fabric properties are dependent both upon the yarn and the type of knit. Underwear, sweaters, socks, sport shirts, sweat shirts, etc. are generally derived from knit fabrics.

Non-woven fabrics are sheets of fabric made by bonding and/or interlocking fibers and filaments by mechanical, thermal, chemical or solvent mediated processes. The resultant fabric can be in the form of web-like structures, laminates or films. Typical examples are disposable baby diapers, towels, wipes, surgical gowns, fibers for the “environmental friendly” fashion, filter media, bedding, roofing materials, backing for two-dimensional fabrics and many others.

The textile used in the present invention may be any known textile (woven, knitted, or non-woven). In particular the textile may be a cellulose-containing or cellulosic textile, such as cotton, viscose, rayon, ramie, linen, lyocell (e.g., Tencel, produced by Courtaulds Fibers), or mixtures thereof, or it may be a synthetic textile such as one of polyester, polyamic or nylon or mixtures of these or a mixture of cellulose-containing or cellulosic fibres and synthetic fibres. Another example of a suitable textile is one comprising other natural fibers such as wool and silk or mixtures of these or mixtures of these and one or more of the above mentioned fibres. Examples of mixtures of fibres include but are not limited to viscose/cotton blends, lyocell/cotton blends, viscose/wool blends, lyocell/wool blends, cotton/wool blends; flax (linen), ramie and other fabrics based on cellulose fibers, including all blends of cellulosic fibers with other fibers such as wool, polyamide, acrylic and polyester fibers, e.g. cotton/polyester blends, viscose/cotton/polyester blends, wool/cotton/polyester blends, flax/cotton blends etc. The term “wool,” means any commercially useful animal hair product, for example, wool from sheep, camel, rabbit, goat, llama, and known as merino wool, Shetland wool, cashmere wool, alpaca wool, mohair, etc. and includes wool fiber and animal hair. The textile may be bleached, dyed or undyed. The term “polyester” refers to poly(ethylene terephthalate) which is synthesized by condensation, drawn into fibers from a melt, possibly cut to stables, possibly mixed with other fiber types, and spun to yarn. The yarn is dyed and knitted into cloth or made into carpets, or the yarn is woven into fabric and dyed.

The ability of an enzyme to bind or adhere to a textile depends both on the particular enzyme but also on the type of textile. For example lipolytic enzymes appear to be more difficult to remove from a polyester-textile than from a cotton-textile during rinsing, in general the adherence or binding of lipolytic enzymes to hydrophobic materials may be stronger than to more hydrophilic materials.

### Further Substances

In a particular embodiment of the present invention the textile may further comprise other substances, such as a protein, lipid, saccharide or a mixture of these. In particular such further substances may be similar to the stains that people get on their clothes in their everyday-life. Examples of such substances that people generally experience as stains on their clothes include but are not limited to grass, mud, clay, coffee, tea, blood, egg, lard, moulds (damp stained) or substances which have been processed, such as butter, processed meat, dyed lard, oil, make up, spice blends, processed tomatoes (ketchup or puree), chocolate, ice cream, cacao, baby food and the like. The textile may also comprise a man made composition comprising compounds selected from refined protein compositions, refined polysaccharide compositions, refined fatty acid compositions, refined triglyceride compositions or other refined biological or non-biological compounds. Another example of a suitable further substance include a particulate composition such as carbon particles, e.g. carbon black or iron oxides. The textile may be stained by applying the staining material as it is or as an aqueous solution onto the textile surface by soaking, brushing and/or spraying. The stained textile may typically be dried before use. Textiles comprising a range of different stains/substances are commercially available under the trade name EMPA® swatches marketed by EMPA St. Gallen, Lerchfeldstrasse 5, CH-9014 St. Gallen, Switzerland.

The inventors of the present invention believe that some of these further substances may form a matrix together with the textile in which an enzyme may be “trapped” during washing, which makes it difficult to remove it during rinsing.

Examples of proteins which may be present at the textile includes but are not limited to proteins present in milk, meat, egg, blood or other proteins which may be present in one of those substances and/or compositions mentioned above which the textile may be stained with.

The term “lipid” is in the context of the present invention to be understood as a group of compounds, which are insoluble in water but soluble in organic solvents such as ether, acetone and chloroform. Said group includes fats, oils, triacylglycerols, fatty acids, glycolipids, phospholipids and steroids. Fatty acids are simple lipids which comprise a carboxylate group at the end of an (often long) hydrocarbon chain with the general formula of CH3(CₓH_{y})COOH and they are constituents of more complex lipids. Triacylglycerols are triesters of fatty acids and glycerol, where the fatty acids in a triacylglycerol may be identical but in many triacylglycerols they are different. Fats are substances which generally comprise a mixture of triacylglycerols and fatty acids and which are solid at 20° C. Oils are similar to fats with the exception that they are liquid at 20° C., because they comprise a higher content of unsaturated fatty acids than the fats. The composition of fats and oils is generally described by their composition of fatty acids, both those present in triacylglycerols and those which are free fatty acids. Glycolipids are lipids comprising a saccharide group. Phospholipids are lipids which comprise a phosphate group in the hydrophilic part of the compound. Steroids are a group of compounds which include cholesterol and sex hormones of higher animals and cholesterol is the precursor for synthesis of many of these substances in nature.

In a particular embodiment of the present invention the lipid may be a “stain-causing lipid”, i.e. a lipid or mixture of lipids which are often the cause of stains on the clothes in the everyday life, examples of such lipids include but are not limited to olive-oil, butter, lard, milk fat, lipstick, vegetable fats or beef fat. The composition of such lipids is often described by their content of fatty acids.

### Contacting an Enzyme with a Textile

The enzyme and the library of polypeptides may be contacted with the textile by any means. In particular this may be performed by adding a solution of the enzyme or library of polypeptides to the textile.

For example if the enzyme or library of polypeptides are expressed and secreted by a host cell, the supernatant from the host cell may be added to the textile, or if the enzyme or the library of polypeptides are expressed inside the host, e.g. in inclusion bodies, the host cells may be lysed and the lysate or a fraction thereof may be added to the textile.

The enzyme or library of polypeptides may also be purified before contacting them with the textile. In this context the term “purified” means that the enzyme/library of polypeptides has been removed from their native environment. If the enzyme/library of polypeptides has been expressed by a host cell the native environment refers to the host cells and compounds different from the enzyme/library of polypeptides secreted by the host cell. For enzymes or polypeptides which have been prepared synthetically this may refer to the removal of other components which have been present during the synthesising process. The enzyme or library of polypeptides may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989).

In a particular embodiment the enzyme or library of polypeptides may be contacted with the textile by adding a detergent-solution comprising the enzyme or library of polypeptides to the textile. The term “detergent-solution” is in the context of the present invention to be understood as a solution comprising one or more surfactants, which may be non-ionic including semi-polar and/or anionic and/or cationic and/or zwitterionic. The surfactants are typically present at a level of from 0.1% to 60% by weight.

The detergent-solution may contain from about 1% to about 40% of an anionic surfactant such as linear alkylbenzenesulfonate, alpha-olefinsulfonate, alkyl sulfate (fatty alcohol sulfate), alcohol ethoxysulfate, secondary alkanesulfonate, alpha-sulfo fatty acid methyl ester, alkyl- or alkenylsuccinic acid or soap.

The detergent-solution may usually contain from about 0.2% to about 40% of a non-ionic surfactant such as alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, or N-acyl N-alkyl derivatives of glucosamine (“glucamides”).

The detergent-solution may further contain 0-65% of a detergent builder or complexing agent such as zeolite, diphosphate, triphosphate, phosphonate, carbonate, citrate, nitrilotriacetic acid, ethylenediaminetetraacetic acid, diethylenetriaminepentaacetic acid, alkyl- or alkenylsuccinic acid, soluble silicates or layered silicates (e.g. SKS-6 from Hoechst).

The detergent-solution may further comprise one or more polymers. Examples include but are not limited to carboxymethylcellulose, poly(vinylpyrrolidone), poly(ethylene glycol), poly(vinyl alcohol), poly(vinylpyridine-N-oxide), poly(vinylimidazole), polycarboxylates such as polyacrylates, maleic/acrylic acid copolymers and lauryl methacrylate/acrylic acid copolymers.

The detergent-solution may further contain a bleaching system which may comprise a H₂O₂ source such as perborate or percarbonate which may be combined with a peracid-forming bleach activator such as tetraacetylethylenediamine or nonanoyloxybenzenesulfonate. Alternatively, the bleaching system may comprise peroxyacids of e.g. the amide, imide, or sulfone type.

The detergent-solution may further comprise conventional enzyme-stabilizing agents, e.g., a polyol such as propylene glycol or glycerol, a sugar or sugar alcohol, lactic acid, boric acid, or a boric acid derivative, e.g., an aromatic borate ester, or a phenyl boronic acid derivative such as 4-formylphenyl boronic acid, and the composition may be formulated as described in e.g. WO 92/19709 and WO 92/19708.

The detergent-solution may also contain other conventional detergent ingredients such as e.g. fabric conditioners including clays, foam boosters, suds suppressors, anti-corrosion agents, soil-suspending agents, anti-soil redeposition agents, dyes, bactericides, optical brighteners, hydrotropes, tarnish inhibitors, or perfumes.

Furthermore, the detergent-solution may comprise one or more other enzyme than the enzyme or enzyme of interest of the present invention, such as a protease, a lipase, a cutinase, an amylase, a carbohydrase, a cellulase, a pectinase, a mannanase, an arabinase, a galactanase, a xylanase, an oxidase, e.g., a laccase, and/or a peroxidase. Examples of enzymes generally used in detergents are well-known to a person skilled in the art.

In a particular embodiment of the present invention mechanical stress may be used when the textile or the enzyme or library of polypeptides is contacted with the textile. In particular contacting the enzyme or library of polypeptides with the textile may be performed as described in WO 02/42740 which discloses a method for testing the cleaning effect of a compound or composition thereof.

### Rinsing the Textile

In the context of the present invention the term “rinsing” is to be understood as contacting the fabric with a water-based solution and subsequently removing said solution, wherein the term “water-based solution” is to be understood as a solution of water comprising a maximum of 20 w/v % other components than water, such as a maximum of 10 w/v % or 5 w/v % or 3 w/v % or 2 w/v % or 1 w/v % or 0.5 w/v % other components than water. Examples of such other components are given below.

As the methods of the present invention may be used to mimic the process of washing clothes the rinsing may in particular mimic the conditions of rinsing during washing of clothes. Generally, clothes are rinsed with water; however the composition of the water may vary depending on the source of the water, e.g. it may be river water, spring water or ground water. Furthermore, the geographical location of the source of water may affect its composition.

The hardness (total hardness) of a given source of water is due to its content of salts of the alkaline earth metals: calcium, magnesium, strontium and barium. Since strontium and barium are generally present in water only in traces, the hardness of water is defined as the content of calcium ions (Ca²+) and magnesium ions (Mg²+). The conventional procedure is to relate the statement of the water hardness only to calcium, in other words to express also the content of magnesium ions as calcium content. A practical measurement unit for the hardness that is frequently employed is the so-called German degree, which is defined as follows
1° dH=10 mg CaO/liter

“Hard” water is water that contains high concentrations of calcium carbonate and other minerals.

“Soft” water is water that contains low concentrations of calcium carbonate and other minerals.

Examples of other components which may be present in the water-based solution used for rinsing include but are not limited to buffers, especially buffers with a pH between 4-10, salts, softeners and small amounts of detergent. There may in particular be small amounts of detergent present if contacting the textile with the enzyme/library of polypeptides has been performed in the presence of a detergent, e.g. by “washing” of the textile. Examples of suitable buffers include but are not limited to those described below in table 1.

TABLE 1

| pKa (20° C.) | Buffer | pH range |
|---|---|---|
| 6.15 | MES | 5.5-7.0 |
| 6.46 | Bis-Tris | 5.7-7.3 |
| 6.6 | ADA | 5.8-7.4 |
| 6.8 | PIPES | 6.1-7.5 |
| 6.9 | ACES | 6.0-7.5 |
| 6.95 | MOPSO | 6.2-7.4 |
| 6.15 | BES | 6.6-8.0 |
| 7.2 | MOPS | 6.5-7.9 |
| 6.5 | TES | 6.8-8.2 |
| 7.55 | HEPES | 6.8-8.2 |
| 7.6 | DIPSO | 6.9-8.1 |
| 7.7 | TAPSO | 7.0-8.2 |
| 7.85 | POPSO | 7.2-8.5 |
| 9.9 | HEPPSO | 7.4-8.6 |
| 8 | EPPS | 7.5-8.5 |
| 8.15 | Tricine | 7.8-8.8 |
| 8.35 | Bicine | 7.7-9.1 |
| 8.4 | TAPS | 7.7-9.1 |
| 9.5 | CHES | 8.6-10.0 |
| 10 | CAPSO | 9.3-10.7 |
| 10.4 | CAPS | 9.7-11.0 |

Examples of salts or ions which may be present in water-based solution besides the Ca²⁺ and Mg²⁺ ions described above include but are not limited to NaCl, KCl, Strontium and barium.

Softeners are generally used during washing to improve the feel and freshness of the clothes and to reduce static electricity buildup (see e.g. Levinson M I, 1999, Journal of Surfactants and Detergents, 2, 223-235). One of the main ingredients in softeners is cationic tensides or surfactants, e.g. diamidoamine or diester quaternary or a triethanolamine-based esterquat, however, it may also comprise other ingredients such as perfumes, preservatives, buffers, dyes, optical brighteners, enzymes, dye stabilizers, ultraviolet light absorbers, chlorine scavengers and/or electrolytes (see e.g. Levinson M I, 1999, Journal of Surfactants and Detergents, 2, 223-235).

### Method for Measuring the Enzymatic Activity

The activity of the residual enzyme/enzyme of interest present at the textile may be measured by any suitable method. The method of choice may depend on e.g. the particular enzyme/enzyme of interest. In the context of the present invention the term “residual enzyme/enzyme of interest” is to be understood as the enzyme/enzyme of interest present at the textile after said textile has been contacted with the enzyme or library of polypeptides and subsequently rinsed according to a method of the present invention. Contacting the enzyme or library of polypeptides with the textile and rinsing it may be performed as described above.

In a particular embodiment the activity of the enzyme/enzyme of interest may be measured by adding a substrate for the enzyme/enzyme of interest which is labelled with a fluorescent compound, wherein the fluorescent label is released from the substrate when it interacts with the enzyme/enzyme of interest. The conversion of substrate to product by the enzyme/enzyme of interest may then be measured by measuring the fluorescence or change in fluorescence. The principles for this method are general and independent of the particular enzyme. Methods for measuring the fluorescence are well known to a person skilled in the art. Other methods may be used including methods which relate more specifically to the particular enzyme/enzyme of interest. Examples of suitable fluorescent molecules with which the substrate may be labelled include but are not limited to Resorufin or Methylumbelliferon.

For example if the enzyme/enzyme of interest is a lipolytic enzyme the substrate may be a fatty acid, such as butyric acid, valeric acid, caproic acid, caprylic acid, capric acid, lauric acid, myristic acid, palmitic acid, stearic acid, arachidic acid, behenic acid, lignoceric acid, cerotic acid, palmitoleic acid, oleic acid, linolenic acid or arachidonic acid. Thus in a particular embodiment of the present invention the enzyme/enzyme of interest may be a lipolytic enzyme and the substrate may be Resorufin-butyrate, Methylumbelliferon-butyrate or Methylumbelliferon-palmitate. Other examples of using fluorescence to measure the activity of lipolytic enzymes include the use of a triacylglycerol where one of the alkyl groups has been substituted with a fluorescent group such as pyrenyl. For example fluorogenic and isomerically pure I-(3)-o-alkyl-2,3-(3,2)-diacylglycerols have been described as useful substrates for measuring the activity of lipases (reviewed in Gupta R et al., Biotechnol. Appl. Biochem (2003), 37, 63-71).

Other suitable substrates include those described by e.g. Gupta R et al., Biotechnol. Appl. Biochem (2003), 37, 63-71, such as Triolein, tributyrin, triacetin (triacetylglycerol), or tripropionin (tripropionylglycerol). Another example is the use of a p-nitrophenyl ester of a fatty acid, e.g. one of the above mentioned fatty acids, for example p-nitropenyl palmitate has been used to measure the activity of lipases.

If the enzyme/enzyme of interest is a protease (E.C. 3.4.) casein or a casein derivative may be used as substrate. In particular the substrate may be casein or a casein derivative labelled with a fluorescent compound, so that the interaction between the protease and casein may be measured by measuring the flourescences. Examples of such fluorescent compound include fluorescein thiocarbamoyl (FTC), BODIPY® FL and BODIPYO TR-X, where the two latter are both compounds obtainable from Molecular Probes, e.g. as part of the EnzCheck® Protease Assay kit. If the protease is a Caspase with a substrate-specificity for the amino acid sequence Asp-Glu-Val-Asp (DEVD) the 7-amino-4-methylcoumarin-derived substrate Z-DEVD-AMC (where Z represents a benzyloxycarbonyl group) may be used as substrate as described in the EnzChek® Caspase-3 Assay kit from Molecular Probes.

If the enzyme/enzyme of interest is an alpha-amylase (E.C. 3.2.1.1) the substrate may be starch or a starch derivative. In a particular embodiment the substrate may be starch obtained from corn labelled with the BODIPY® FL dye which is part of the EnzChek® Amylase Assay kit from Molecular Probes.

If the enzyme/enzyme of interest is a cellulase (E.C. 3.2.1.4) the substrate may be native cellulose, in particular it may be native cellulose labelled with 5-(4,6-dichlorotrazinyl)aminofluorescein (DTAF) as described in Helbert W et al., (2003), Biomacromolecules, 4, 481-487. Another example of a suitable substrate includes the cellhexaose derivative comprising a naphthalene moiety at the reducing end and a 4-(4′dimethylaminobenzeneazo)-benzene at the non-reducing end as described in Boyer V et al, (2002), Chemistry-a European Journal, 8 (6), 1389-1394.

If the enzyme/enzyme of interest is a peroxidase (E.C. 1.11.1) the enzyme activity may be measured by measuring conversion of hydrogen peroxide as a function of time by using an assay based on ABTS® (2,2′-azinobis(3-ethylbenzothiazoline-6-sulfonate)) as the chromophore. The greenish-blue colour of the oxidized ABTS can be measured by a photometer at 418 nm.

### Methods of the Invention

The inventor of the present invention has found that the amount of residual enzyme on a textile may be measured by measuring the activity of said enzyme. Thus the present invention relates in one embodiment to a method for measuring the amount of residual enzyme on a textile comprising measuring the activity of the enzyme, wherein the textile has been contacted with the enzyme and subsequently rinsed prior to measuring the enzyme activity.

In another embodiment the present invention relates to a method for screening a library of polypeptides for an enzyme of interest comprising measuring the amount of residual enzyme present on a textile using above method and then selecting an enzyme.

In the context of the present invention the term “residual” refers to the enzyme which is left on a textile after said textile has been contacted with the enzyme and subsequently rinsed according to a method of the present invention.

For both methods the textile is first contacted with the enzyme, then the textile is rinsed and the activity of the enzyme present at the textile is measured. Thus schematically shown said methods may comprise the steps of:
a) contacting the textile with the enzyme or library of polypeptides
b) rinsing the textile
c) measuring the activity of the enzyme present on the textile,
   wherein the screening method further comprises a step of selecting an enzyme of interest.

As most enzymes have an optimal functionality at temperatures between 5 and 95° C., the methods of the present invention may in particular be carried out at 5-95° C., e.g. 10-80° C., 20-70° C., 20-60° C., 20-50° C.

The methods of the present invention may take place in any suitable container, such as microtiter plates with e.g. 24 wells/plate, 96 wells/plate, 384 wells/plate, 1536 wells/plate or a higher number of wells per plate, or nanoliter well-less compartments. An advantage of using a microtiter plate is that it is generally easy to automate the detection procedure which is particularly useful when a library of polypeptides is screened.

If the methods of the present invention are carried out in a microtiter plate the textile may have the form of a small patch with a size suitable for placement at the bottom of the well in a plate.

The activity of enzyme/enzyme of interest present on the textile may be compared with a control. For example when a library of polypeptides is screened for an enzyme of interest the enzymatic activity present on the textile may be compared with the enzymatic activity of a known enzyme, e.g. if a library of variants is screened it may be compared with the parent enzyme. This may be relevant if one wishes to find a variant with a similar ability to degrade stains on the clothes as the parent enzyme but which is easier to remove from the clothes during rinsing. Thus in this case one would choose a variant for which the amount of residual enzyme on the textile is less than it is for the parent. For example as described in the examples when screening for a lipase known lipases such as Lipolase® and Lipex® may be used as controls.

Another example of using a control is the use of a so-called internal standard which enables one to correct for assay-assay variations. Typically, a well known enzyme showing low activity in the assay and a well known enzyme showing high activity in the assay may be used as internal standards and these are then included in every assay; this shows that the variations in enzymatic activity may be used as an indicator of the assay-assay variations.

It is generally preferred that there are no enzymes present on the clothes after they have been washed with a detergent comprising an enzyme. Thus it would be an advantage to be able to detect how much enzymes there is left on the clothes. For example, if a lipolytic enzyme has been used for washing, it may be an advantage to measure the amount of residual enzyme, as some lipolytic enzymes may be able to react with substrates present on the clothes to release fatty acids which do not smell good.

### MATERIALS AND METHODS

### Enzymes

Lipolase® is a lipase derived from Humicola lanuginosa described in EP 258 068 and EP 305 216.

Lipex® is a variant of Lipolase® and described in WO 0060063.

### Textile-Swatches

wfk20LS is a textile-swatch with Lipstick on Polyester/Cotton 65/35 obtained from wfk Testgewebe GmbH, Christenfeld 10, D-41379 Bruggen, Germany.

### Methods

### Micro-Laundry

Textile swatches stained with lard or butter were punched into wells of a 96 well microtiterplate. 150 μl of detergent (100 mM L-arginine) was dispensed into each well. 10 μl supernatant of yeast cells expressing the enzyme to be tested (grown in microtiterplates for 3-4 days in SC medium) was added to each well and microtiterplates are incubated for 20 min at 30° Celcius at 500 rpm. The wash water was removed by a plate washer and the swatches were subsequently rinsed as described below.

### Rinsing

After performing the micro-laundry assay (described above) with different enzyme concentrations, the textile swatches present in the microwells were rinsed using artificially made water with a hardness of 15° dH (see materials and methods).

The rinsing process was performed by adding 180 microl water with a hardness of 15° dH, placing the plate on a orbital shaker set at 300 rpm for 5 min before removing the rinse water. This rinse process was repeated 3 times in total. After the final removal of rinse water a lipase substrate was added to the wells to measure the activity of lipase present on each textile-swatch.

### EXAMPLES

### Example 1

### Measuring Residual Lipase on a Cotton/Polyester Textile Stained with Lipstick

Textile swatches made of 35% cotton and 65% polyester stained with lipstick (wfk20LS) were washed according to the micro-laundry procedure described above with the detergent comprising different concentrations of either Var1, Var2, Var3, Var4, Var5 or Var6 (which are all variants of Lipolase®), Lipolase® or Lipex®. Lipolase® and Lipex® were used for comparison with the variants. Methylumbelliferon-butyrate (Fluka #19362) was dissolved in water with a hardness of 15° dH containing 0.4% Triton X-100 (Sigma T9284) ending at a Methylumbelliferon-butyrate concentration of 200 microM. 100 microl of this substrate was added to each of the wells comprising a textile swatch and then incubated at room temperature for 1 hour. After 1 hour the fluorescence was measured in the fluorometer SpectraFluorPlus (Tecan, Austria) with the excitation wavelength set to 360 nm and the emission wavelength set to 465 nm. The fluorescence at 465 nm for each concentration and lipase/lipase variants is shown below in table 2:

TABLE 2

| Amount | | | | | | | | |
|---|---|---|---|---|---|---|---|---|
| of | | | | | | | | |
| lipase | | | | | | | | |
| (ppm) | Lipolase ® | Lipex ® | Var1 | Var2 | Var3 | Var4 | Var5 | Var6 |
| 0 | 24262 | 20832 | 20654 | 19137 | 20727 | 20751 | 18404 | 19237 |
| 0.8 | 25319 | 24366 | 22399 | 22660 | 24887 | 20679 | 19183 | 19806 |
| 1.7 | 24735 | 25954 | 26255 | 21922 | 29010 | 23041 | 20513 | 21157 |
| 3.3 | 22054 | 28216 | 30892 | 23219 | 37018 | 34901 | 28483 | 28659 |
| 5 | 23344 | 30281 | 37562 | 26986 | 44024 | 34317 | 32561 | 29697 |
| 8 | 23980 | 36473 | 41684 | 27636 | 46401 | 36608 | 37653 | 32188 |

The results show that the Var1 and Var6, the latter only at some concentrations emits less flourescence than Lipex®), indicating that there are less of those variants present on the textile after wash than there is of Lipex®.

Similar amounts of residual lipase were found by other assays.

### Example 2

### Measuring Residual Lipase on a Cotton/Polyester Textile Comprising Butter and Sudan Red

Textile swatches made of 35% cotton and 65% polyester comprising lipstick (wfk20LS) were washed according to the micro-laundry procedure described above with the detergent comprising different concentrations of either Var1, Var3, Var5, Lipolase® of Lipex®. Resorufin-butyrate (Fluka #83637) was dissolved in water with a hardness of 15° dH containing 0.4% Triton X-100 (Sigma T9284) ending at a Resorufin-butyrate concentration of 2 microM. 100 microl of this substrate was added to each of the wells comprising a textile swatch and then incubated at room temperature for 1 hour. After 1 hour the fluorescence was measured in the fluorometer Polarstar (from BMG Labtechnologies GmbH, Germany) with the excitation wavelength set to 530 nm and the emisson wavelength set to 590 nm. The fluorescence at 590 nm for each concentration and lipase/lipase variants is shown below in table 3:

TABLE 3

| Amount of | | | | | |
|---|---|---|---|---|---|
| lipase (ppm) | Lipolase ® | Lipex ® | Var1 | Var3 | Var5 |
| 0 | 6987 | 6066 | 6487 | 6277 | 6723 |
| 2 | 11018 | 45719 | 46753 | 58403 | 30985 |
| 4 | 15442 | 51096 | 51609 | 63201 | 37562 |
| 8 | 16982 | 54395 | 56677 | 62206 | 43321 |

The results show that the Var5 emits less flourescence than Lipex®, indicating that there is less of that variant present on the textile after wash than there is of Lipex®.

Similar amounts of residual lipase were found by other assays.

1-10. (canceled)

11. A method for measuring the amount of residual enzyme on a textile comprising measuring the activity of the enzyme, wherein the textile has been contacted with the enzyme and subsequently rinsed prior to measuring the enzyme activity.

12. The method of claim 11, comprising the steps of:
a) contacting the textile with an enzyme
b) rinsing the textile
c) measuring the activity of the enzyme on the textile.

13. A method for screening a library of polypeptides for an enzyme of interest comprising
a) measuring the amount of residual enzyme on a textile comprising measuring the activity of said enzyme, wherein the textile has been contacted with the library of polypeptides and subsequently rinsed prior to measuring said activity
b) selecting an enzyme of interest.

14. The method of claim 13, wherein step a) comprises the steps of:
i) contacting the library of polypeptides with a textile
ii) rinsing the textile
iii) measuring the activity of enzyme of interest on the textile.

15. The method of claim 11, wherein the enzyme is selected from the group consisting of oxidoreductase, transferase, hydrolase, lyase, isomerase and ligase.

16. The method of claim 15, wherein the enzyme is a lipolytic enzyme.

17. The method of claim 11, wherein the amount of residual enzyme is less than for a control.

18. The method of claim 11, wherein the textile is made of cotton, polyester, wool or a mixture of any of these.

19. The method of claim 11, wherein the textile further comprises a substance.

20. The method of claim 11, wherein the activity of the enzyme is measured by addition of a fluorescent substrate to the textile comprising the enzyme or library of polypeptides and measuring the amount of fluorescence.
